(19) United States
(12) Patent Application Publication
(10) Pub. No.: US 2010/0318169 A1
(43) Pub. Date: Dec. 16, 2010

(54) DELIVERY SYSTEM FOR ENDOLUMINAL DEVICES

(75) Inventor: Daphne A. Kontos, Washington, DC (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/482,057

(22) Filed: Jun. 10, 2009

(51) Int. Cl. A61F 2/06

(52) U.S. Cl. 623/1.11

(57) ABSTRACT

An introducer or delivery system for introducing implants or medical devices such as embolic protection devices within a patient allows deployment within vessels having a tortuous anatomy. The delivery system includes an actuator wire within the delivery catheter which enables the delivery catheter to be bent near a distal end thereof upon actuation of the actuator wire.

### FIELD OF THE INVENTION

The present invention relates to an introducer or delivery system for intraluminal medical devices, in an example, to a delivery system for embolic protection devices. The taught introducer can also be used for delivering stents, stent grafts, vena cava filters, occlusion devices and other forms of implant, as well as for other procedures such as balloon angioplasty.

### BACKGROUND OF THE INVENTION

Balloon angioplasty and stenting procedures are widely used in the treatment of stenoses of the coronary arteries as an alternative to invasive bypass surgeries. However, the inflation of a balloon or placement of a stent at the stenosed region can dislodge embolic particles from the lesion that may travel downstream (distal) of the stenosis. In certain critical arteries, such as carotid arteries, the embolic particles may become trapped in small-diameter blood vessels of the brain and may cause a stroke.

To increase the safety of carotid angioplasty and stenting procedures, embolic protection devices have been developed as a means to capture embolic particles that have been dislodged from a stenosis. Such devices may be deployed within a vessel at a site distal of the stenosis before the angioplasty or stenting procedure takes place. In a deployed configuration, the embolic protection device is intended to act as a filter that allows blood to pass but traps embolic particles traveling downstream.

For example, an embolic protection device may be attached to a wire guide and encased within a sheath, and then loaded into a guiding catheter for delivery to a site proximal of the stenosis. A clinician may advance the embolic protection device and the sheath surrounding it out of the distal end of the guiding catheter and across the stenosed region by pushing on the wire guide. Once the device is positioned at a site distal of the stenosis, the clinician may remove (e.g., retract) the sheath to deploy the embolic protection device to an expanded configuration for use. The embolic particles trapped in the expanded device may be removed from the vessel by collapsing the device and retracting the wire guide.

In one particular current method, an introducer is used to gain access to the patient's vascular system at a location proximal to the treatment site. Either a guidewire or embolic protection device (EPD) is advanced through the introducer to the treatment site. Stent and balloon systems later follow the guidewire or EPD once this is in place. The EPD is either mounted on its own delivery system or compatible with a range of guidewires.

The narrowed vessel may be hard to reach and cross due to its location. This may be due to an almost total occlusion or complicated branch point where the desired vessel is set at a steep angle to the current path the guidewire or EPD is travelling. Extra support and proper angling is needed in order to cross the narrowed vessel.

In fact, in about 20-30% of carotid stenting situations, the anatomy of the vessel may be too tortuous to permit placement of the embolic protection device in the above-described manner using existing delivery systems. A tortuous vessel may contain severe bends, kinks or coils that can inhibit delivery of the embolic protection device.

### SUMMARY OF THE INVENTION

The present invention seeks to provide an improved delivery system or introducer.

The delivery system may be for an embolic protection device. The delivery system may allow the device being delivered to be transported and deployed within vessels having a tortuous anatomy.

According to an aspect of the present invention, there is provided an introducer for delivering an implant or other medical device within a patient, comprising a delivery catheter including a distal end and a proximal end the catheter including a lumen for receiving an implant or medical device for delivery and an actuator lumen; and an actuator device located within the actuator lumen and operable to cause deflection of the catheter upon deployment of the actuator device; wherein the actuator device is an elongate flexible actuator element fixed to or proximate a distal end of the delivery catheter and able to be pulled in a proximal direction so as to cause deflection of the delivery catheter.

According to another aspect of the present invention there is provided an introducer for delivering an implant or other medical device within a patient, comprising a delivery catheter including a distal end and a proximal end, the catheter including a delivery lumen for receiving an implant or medical device for delivery and an actuator lumen; an elongate actuator wire located within the actuator lumen at a position offset from a centre axis of the delivery catheter and fixed at one of at or proximate the distal end of the delivery catheter and extending beyond the proximal end of the delivery catheter; the elongate actuator element being able to be pulled in a proximal direction from the proximal end of the delivery catheter so as to cause deflection of the delivery catheter.

In order to guide through tortuous vasculature, the preferred embodiments provide a torqueable shaft. The shaft gives the user control of the device, allowing more precise guiding. Part of the main shaft provides a lumen for injection/aspiration purposes. The injection of contrast into the vessel through that lumen allows for the user to view the location of the delivery tip under radiography. Attached to the shaft there may be provided a free working lumen where an accessory device can be loaded for delivery. These accessory devices lack superior torque control and flexibility. The described embodiments simplify delivery of those devices in hard to reach treatment sites.

The preferred embodiment provides a catheter, a shaft and an actuating mechanism. The actuating mechanism is attached to the distal end of the catheter and is preferably made of a free activating wire or a coil with inner actuating wire combination. The tip deflecting action takes place at the distal end of the catheter by applying tension to the activating wire which is controlled at the proximal end of the catheter. There may be provided a locking mechanism to fix the catheter tip at a specific angle. The device will have an open lumen spanning the length of the catheter used for injection purposes.

The catheter shaft may be braided, stranded or have a cannula tubing that extends distally to the ultimate length minus 2 to 5 cm in some embodiments. Encased in the shaft are the actuating lumen and injection lumen which extend distally to the end of the catheter. The distal 20 cm or less of the catheter may include a working lumen large enough to hold an accessory device. The working lumen can be bonded to the main shaft or can have an additional lumen where the shaft components are inserted. The lumen is preferably of a size to accept 4 French devices. The device may be used through a pre-placed 6 French sheath or on its own.

The tip deflecting mechanism allows for proper angling of accessory devices in complex anatomy. Furthermore, the preferred embodiments preferably have a short loading length for the accessory devices, making it easy to remove once the accessory device is in place.

The preferred embodiments can be used in catheterization laboratory by interventional cardiologists. They can be used to access the internal and external carotid arteries or other complicated branch point vessels or to navigate through tortuous vasculature. The taught devices can be used to advance embolic protection devices or balloon catheters or stent systems or other accessory device through difficult to navigate vessels to treatment site. They can also be used in patients who require intervention for diagnosis or treatment procedures, specifically due to narrowing of a vessel by plaque buildup.

### DESCRIPTION OF THE DRAWINGS

Embodiments of the present invention are described below, by way of example only, with reference to the accompanying drawings, in which:

FIG. 1 is a schematic of the human body showing intraluminal access to the left common carotid artery through the femoral artery;

FIG. 2 is a schematic of the aortic arch and a stenosed region in the left common carotid artery;

FIG. 3 is a perspective view of an embodiment of delivery catheter or introducer;

FIG. 4 is a transverse cross-sectional view of the catheter of FIG. 3;

FIGS. 5 to 7 are transverse cross-sectional views of different configurations of introducer or delivery catheter;

FIGS. 8 to 10 show perspective views of parts of another embodiment of delivery catheter or introducer;

FIGS. 11 and 12 show portions of two embodiments of delivery catheter for use in a rapid exchange system;

FIG. 13 schematically shows the positioning of the delivery system within a vessel in preparation for delivery and deployment of the embolic protection device;

FIG. 14 schematically shows the embolic protection device being advanced across the treatment site; and

FIG. 15 schematically shows an embolic protection filter in a deployed configuration.

### DESCRIPTION OF THE PREFERRED EMBODIMENTS

Described herein is an intraluminal delivery system 5 that may be suitable for directing and delivering an embolic protection device 10 to a site distal of a stenosis in a tortuous vessel. The delivery system 5 may advantageously allow carotid stenting procedures to be carried out under a wider range of circumstances.

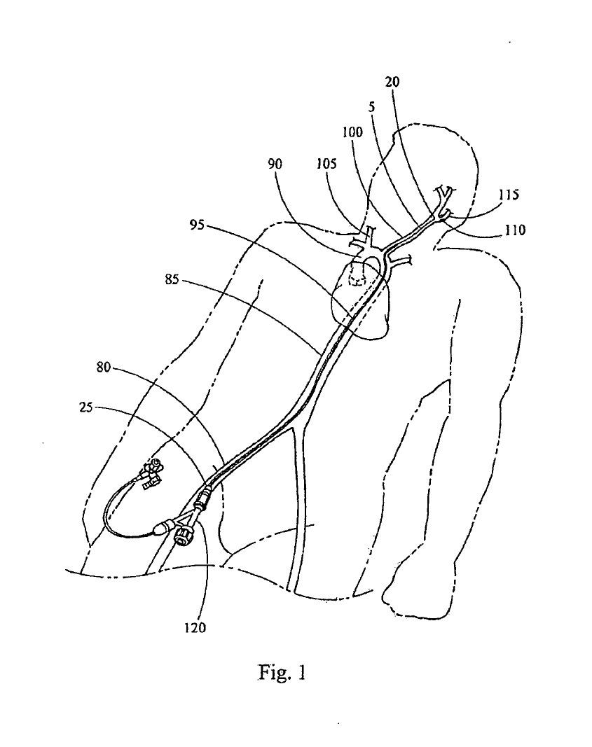

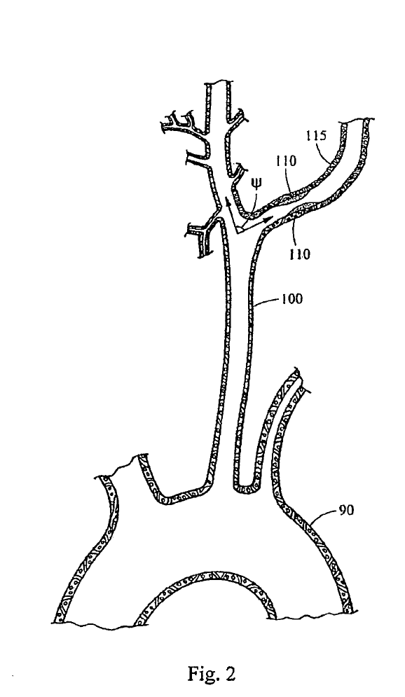

For example, the intraluminal delivery system 5 may be used to deliver an embolic protection device 10 to a treatment site (e.g., a stenosed region) in a tortuous carotid artery. Typically, to access a carotid artery, a percutaneous incision is made in the femoral artery 80 and an intraluminal medical device is advanced through the aorta 85 and the aortic arch 90. This access route is shown schematically in FIG. 1. Branching from the aortic arch 90 are the left common carotid artery 100 and the right common carotid artery 105, which supply blood to the head and neck, as shown in FIG. 2. A stenosed region 110 is shown in the internal carotid artery 115 extending from the left common carotid artery 100 at a take-off angle of ψ. A guiding sheath 95 may be placed within the carotid artery of interest prior to insertion and delivery of the intraluminal medical device, as shown in FIG. 1 and as will be further discussed below. Alternatively, other vascular pathways besides the femoral artery, such as, for example, a radial or brachial artery, may be employed to access the carotid artery of interest.

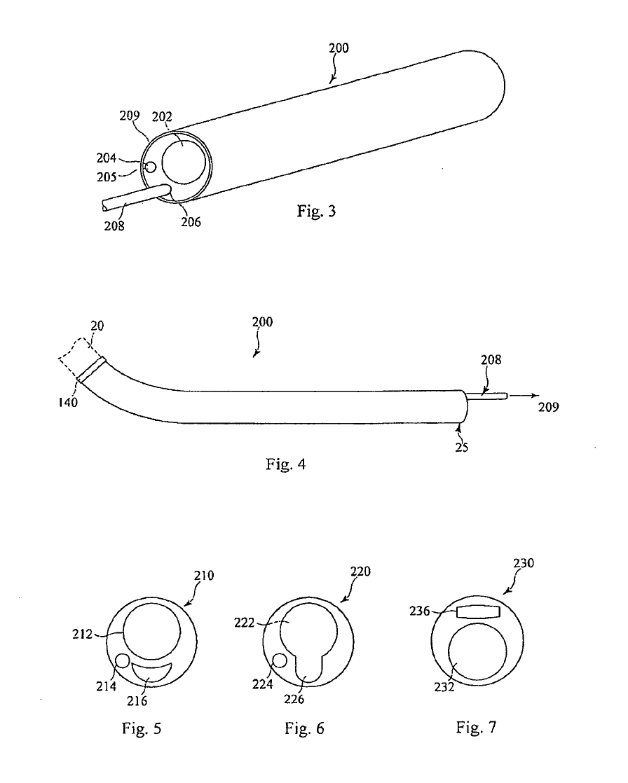

Referring now to FIG. 3, there is shown a first embodiment of delivery catheter 200 for use with the endoluminal delivery system 5 shown in FIG. 1. The catheter 200, which in the preferred embodiments is made from conventional materials, includes in this embodiment three lumens 202, 204 and 206.

The larger lumen 202 is a device delivery lumen for delivery of an embolic protection device (EPD), a balloon catheter, a stent system or any other accessory device, prosthesis or implant to be delivered within a patient. The dimensions of the delivery lumen 202 are dependent upon the particular medical application but would typically be in the region of 3 to 4 French.

The lumen 204 is a guidewire lumen for receiving a guidewire of conventional form.

Finally, the lumen 206 is an actuator lumen for receiving actuator 208, which is typically a flexible metal wire of any suitable metal or alloy, specific examples would be common general knowledge to the skilled person. The actuator 208 extends through the catheter 200 and is fixed at its distal end 20 or at any location adjacent the distal end 20 of the catheter 200. The fixing may be by any suitable mechanism such as by welding, bonding, adhesive, provision of an enlarged end to the actuator wire 208 and so on. Apart from its fixed end, the actuator 208 is able to slide within the actuator lumen 206. As will be appreciated in FIG. 3, the actuator lumen 206 and the actuator wire 208 are offset relative to the centre axis of the catheter 200. As a result of this and of fixing of the distal end of the actuator wire 208 to the distal end of the delivery catheter 200, the application of a tensile force in a proximal direction 209, shown by the arrow in FIG. 4, will compress that side of the delivery catheter 200 adjacent the actuator wire 208 thereby causing the guidewire catheter 200 to flex in a manner similar to that shown in FIG. 4. In practice, as described below, this is achieved by the surgeon pulling on a proximal end of the actuator wire 208 in a direction out of the patient. This provides the delivery catheter 200 with the ability to be steered, thereby substantially facilitating the passage of the delivery catheter 200, particularly through tortuous vasculature.

The catheter 200 in some applications may be provided with a support sheath 209, such as a Myla catheter of known form. Similarly, there may be provided on or adjacent the outer part of the catheter 200 a strengthening element 209 such as a coil, mesh or braid for improved pushability and torquability. The wire of such a coil, mesh or braid could be flat or round and some of the embodiments may have a helical configuration.

In the preferred embodiment, the actuator wire 208 is fixed between 0.5 to 2 cm from the distal end of the catheter 200. However, this may be placed precisely at the distal end or any other position close to the distal end 20 of the delivery catheter 200, in dependence upon the location where it is desired to have most flexure of the catheter 200 during the deployment operation.

In practice, the actuator wire 208 can provide deflection of the tip of the delivery catheter 200 of up to 50 to 80°.

According to one embodiment, the embolic protection device 10 may include an embolic protection filter disposed within delivery sheath 200. Suitable filters may include, for example, Angioguard™ RX, a product of Cordis (Miami Lakes, Fla.); RX Accunet™, a product of Guidant (Indianapolis, Ind.); FilterWire EZ, a product of Boston Scientific (Natick, Mass.); and EmboShield, a product of Abbott Vascular Devices (Redwood City, Calif.). Depending on the filter design, a wire guide may be attached to the filter at the proximal end thereof, or the filter may pass over the wire guide during delivery of the device. Of the representative filters mentioned above, the EmboShield device is an “over-the-wire” device, while the others are “fixed-wire” filters.

FIG. 5 shows another embodiment of delivery catheter 210 which is equally provided with a delivery lumen 212, a guidewire lumen 214 and an actuator lumen 216. This embodiment differs from that of FIG. 3 in that the actuator wire lumen 216 has a moon shape in transverse cross-section and would accommodate an actuator wire of similar transverse configuration. Such a shape of actuator wire and actuator lumen 216 improves torqueability of the delivery actuator 210 and also enables the actuator wire to have a greater cross-sectional area, compared to an actuator wire of round cross-section, for a given outer diameter of delivery catheter 210.

FIG. 6 shows another embodiment of delivery catheter 220 which is also provided with a delivery lumen 222 and a guidewire lumen 224. This embodiment differs from that of FIG. 3 in that the delivery device lumen 222 is provided with an actuator lumen in the form of a recessed area 226 for receiving an actuator wire 208. In other words, there is not a separate actuator wire lumen as in the embodiments of FIGS. 3 and 5.

The embodiment of FIG. 7 shows a delivery catheter 230 having a delivery device lumen 232 and an actuator lumen 236. Although not shown in FIG. 7, the delivery catheter 230 preferably also includes a guidewire lumen. In this embodiment, the actuator wire lumen 236 has a generally rectangular transverse cross-section for improving torqueability of the delivery catheter 230. As with the embodiment of FIG. 5, this embodiment of FIG. 7 can also provide an actuator wire of greater transverse cross-sectional area compared to a device having a round transverse cross-section guidewire for a given size of delivery catheter.

In the embodiments of FIGS. 3 to 7, and indeed in all the embodiments of delivery catheter disclosed herein, delivery catheter may include one or more radiopaque markers 140 near the distal end. The radiopaque markers 140 may be thin-walled tubular structures formed from radiopaque materials, such as, for example, gold, tungsten, platinum, palladium, or alloys thereof. The radiopaque markers 140 may be secured about the circumference of the delivery catheter 200 to improve the visibility of the catheter 200 during non-invasive imaging procedures, such as x-ray fluoroscopy.

According to one embodiment, the delivery catheter 200-230 may include at least two radiopaque markers 140. For example, three, four, five, six or more radiopaque markers 140 may be used. The markers 140 may be spaced at a predetermined distance along the catheter 200-230 and used for calibrating distances during imaging procedures. For example, five markers 140 spaced along the delivery catheter 200-230 a distance of 1 cm apart may be used to calibrate a 4 cm distance in an image.

The radiopaque markers 140 may be secured to the delivery catheter 200-230 by, for example, applying an axial tensile force to the catheter 200-230 to cause a tensile expansion and a radial contraction thereof, and then sliding the one or more markers 140 over the catheter 200-230 before releasing the force. Upon release of the force, the catheter 200-230 may radially expand, and the radiopaque markers 140 may be secured about the circumference of the catheter 15.

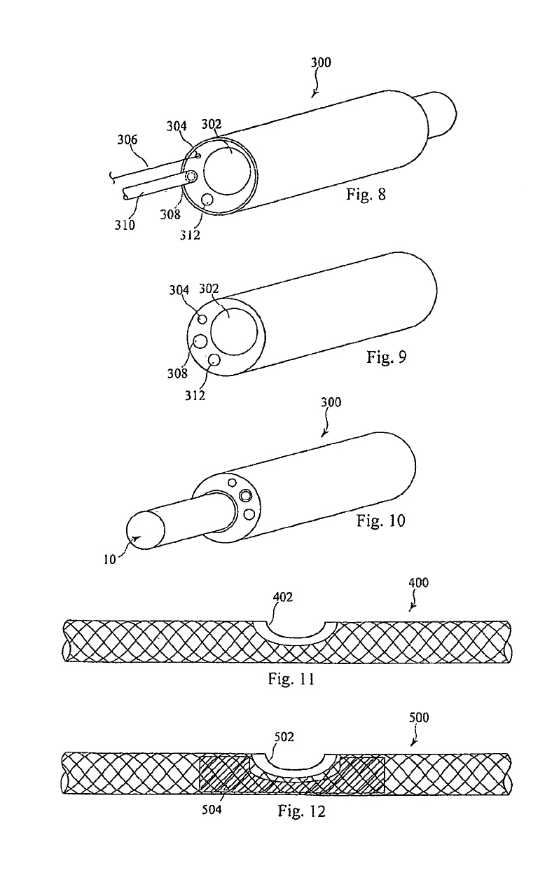

Referring now to FIGS. 8 to 10, there is shown another embodiment of delivery catheter 300. As with the embodiment of FIG. 3, the delivery catheter 300 of FIGS. 8 to 10 includes a delivery device lumen 302, a guidewire lumen 304 for receiving a guidewire 306, an actuator wire lumen 308 for receiving an actuator wire 310. This embodiment differs from that of FIG. 3 by provision of an additional lumen 312. This latter lumen 312 is designed to deliver to the distal end of the delivery catheter 300 a suitable contrast agent for assisting in the positioning of the delivery catheter 300 and the deployment of the device delivered thereby. Any suitable contrast agent of a type well known in the art may be fed through the lumen 312.

Referring now to FIGS. 11 and 12, there are shown two examples of braiding 400, 500 for use as support to the delivery catheter 200-300 shown in FIGS. 3 to 10 and specifically designed to provide a rapid exchange facility to the catheters shown in these Figures. The braiding may be extruded with the catheter and is provided with an aperture 402, 502 close to the distal end of the delivery catheter for rapid exchange purposes known in the art. It is preferred, shown in FIG. 12, that there is provided a support element at the aperture of the braiding, the support element 504 conveniently being a reinforced wire or tube, which may, for example, be made of stainless steel. This reinforces the rapid exchange aperture in a convenient manner.

The reinforcement structure for the delivery catheter 200-300 may be made of a biocompatible metal or alloy, such as stainless steel, and may be embedded within the catheter wall and take the form of a wire, braid, mesh, coil or other arrangement. Such reinforcement structures are known, as are methods of manufacturing medical devices including such structures. Examples can be found, for instance, in U.S. Pat. Nos. 5,700,253 and 5,380,304, the contents of which are incorporated herein by reference.

The embedded reinforcement structure may be exposed or embedded within the wall of the catheter 200-300 and extend along at least a portion of the length of the catheter.

A method for delivering an embolic protection device to a site distal of a stenosis in a tortuous vasculature is now described.

First, a clinician may perform a carotid angiography procedure to obtain a map of the vasculature. The procedure may entail inserting a flush catheter into the common carotid artery 100 and injecting a contrast fluid or dye which is visible under x-ray irradiation. The resulting pictures, called angiograms, allow the clinician to visualize the area and measure the take-off angles of the arteries of interest. Depending on the geometry and configuration of the vessels, use of the delivery catheter 200-300 may be advantageous. If, for example, the take-off angle ψ of the internal carotid artery 115 which contains a stenosed region 110 is greater than about 45 degrees with respect to the common carotid artery 100, as shown in FIG. 2, use of the delivery catheter 15 may be advisable.

Next, the embolic protection device 10 to be used in the procedure may be prepared according to the manufacturer's instructions and then front-loaded or back-loaded into the delivery catheter 200-300. During front-loading of the embolic protection device 10 into the delivery catheter 200-300, the collapsed filter 35 contained within the sheath 40 may be advanced within the delivery catheter 200-300 in a distal direction. For example, the delivery catheter 200-300 may be flushed with saline and the embolic protection device 10 may be advanced through the catheter 200-300 from the proximal end 25 to the distal end 20. In the case of a monorail or rapid exchange catheter design, the embolic protection device 10 may be loaded into the delivery catheter 200-300 through an exit port 402, 502 positioned between the distal and the proximal ends 20, 25. Alternatively, the embolic protection device 10 may be back-loaded into the delivery catheter 200-300. In this case, the device 10 may be loaded into the delivery catheter 200-300 in a proximal direction through the distal end 20.

In the loaded configuration, a portion of the embolic protection device 10 may protrude from the distal end 20 of the delivery catheter 200-300. For example, a distal tip or nose cone of the embolic protection device 10 may protrude from the distal end 20 with the rest of the embolic protection device 10 preferably disposed within the delivery catheter 200-300. Alternatively, the embolic protection device 10 may be contained entirely within the delivery catheter 200-300 for delivery to the treatment site.

Prior to delivery of the embolic protection device 10 to the treatment site using the delivery catheter 200-300, guide wire 205 is placed into the common carotid artery 100 for use as a channel to access the stenosed region.

Once the guide wire 205 or other device has been placed in the common carotid artery 100, the delivery catheter 200-300, including the embolic protection device 10, may be advanced through the arch 90 and the guide wire 205 to a site in the common carotid artery 100 proximal of the stenosis 110. The process may be guided by fluoroscopy, that is, the x-ray tracking of one or more radiopaque markers attached to the delivery catheter 200-300 and/or the embolic protection device 10.

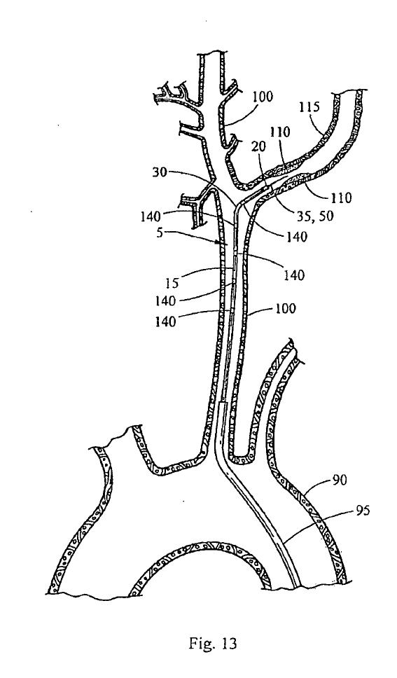

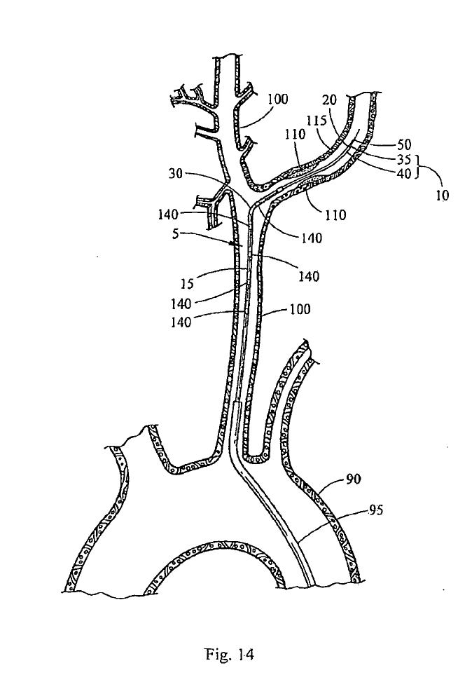

Once the embolic protection device delivery system 5 has been placed in the common carotid artery 100, the actuator wire 208 of the delivery catheter 200-300 may be pulled in a proximal direction to generally align the distal end 20 of the catheter 200-300 in the direction of the artery 115 that contains the stenosis 110. For example, guided by fluoroscopy, the delivery catheter 200-300 may be rotated and maneuvered such that the distal end 20 is positioned in alignment with the left internal carotid artery 115, as shown schematically in FIG. 13. This ability to aim the distal end 20 of the embolic protection device delivery system 5 in this fashion may be particularly advantageous in accessing and traversing tortuous vessels.

Depending on the location of the stenosed region 110 within the artery 115, the embolic protection device 10 may at this point be ejected from the distal end 20 of the delivery catheter 200-300 in the direction of the treatment site 110 in preparation for deployment. Alternatively, the delivery catheter 200-300 itself may be directed into the artery 115 to obtain closer access to the treatment site 110 before advancing the embolic protection device 10 out of the distal end 20. In the latter case, the curvature of the delivery catheter 200-300 may be further exploited to direct the distal end 20 of the delivery system 5 in the desired direction when additional tortuosity is encountered within the vessel. The reinforcement structure disposed within the catheter wall according to some embodiments may further aid in traversing tortuous regions by increasing the pushability and/or torqueability of the delivery catheter 200-300. Preferably, the distal end 20 of the catheter 200-300 is positioned proximal of the stenosed region 110 and does not cross the stenosed region 110.

The embolic protection device 10 is ejected from the distal end 20 of the delivery catheter 200-300 by conventional means. It is be desirable to keep the delivery catheter 200-300 substantially stationary when the embolic protection device 10 is being ejected from the distal end 20 of the catheter 200-300 and across the stenosed region 110, as shown in FIG. 14.

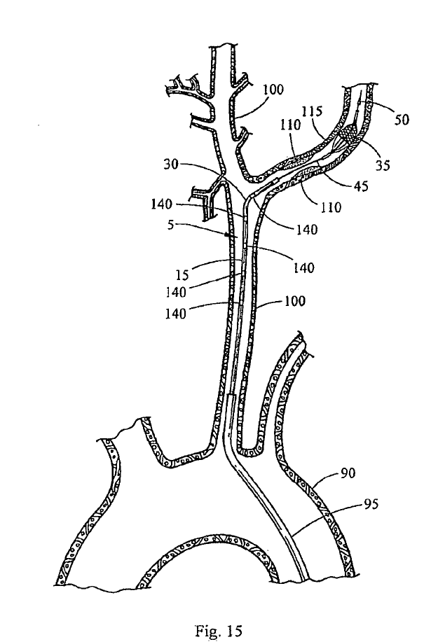

The embolic protection device 10 may be deployed at a site distal of the stenosed region 110 upon which, in the embodiment shown, the filter 35 expands to a deployed configuration within the artery 115. In the deployed configuration, which is shown in FIG. 15, the filter 35 may be able to trap embolic particles generated during an angioplasty procedure and/or deployment of an expandable stent at the stenosed region 110.

The delivery catheter 200-300 may be removed from the patient's body after delivery and/or deployment of the embolic protection device 10. The rapid exchange (or “monorail”) design described above may simplify the process of removing or retracting the catheter 200-300. Preferably, the distal positioning of the wire guide and filter remain substantially unchanged during the retraction. To maintain the internal positioning of the wire guide and filter as the catheter is retracted, a length of wire guide corresponding to the length within the catheter lumen preferably extends outside the patient's body. Consequently, when a catheter overlies a wire guide over its entire length in an over-the-wire configuration, a substantial length of wire guide extends outside of the patient's body. In contrast, in the case of a catheter having a rapid exchange design, the wire guide exits the lumen at an exit port in an intermediate region between the distal and proximal ends, and thus a substantially shorter length of the catheter overlies the wire guide. A shorter length of wire guide may extend outside the patient's body, and the retraction process may be considerably simplified. A single clinician may be able to exchange out the catheter without assistance, whereas a medical assistant may be needed during retraction of an over-the-wire catheter.

If desired, the delivery catheter 200-300 may later be reinserted into the artery 115 to collapse and retrieve the embolic protection filter 35 after completion of the procedure. This may be particularly advantageous due to the torqueability of the delivery catheter 200-300. In some cases, as a recovery catheter is being retracted with the collapsed filter inside, the recovery catheter, which is relatively soft, may become trapped by the open cells of the deployed stent. In such a situation, the torqueability of the present delivery catheter 200-300 may prove advantageous to free the catheter 200-300 from the stent and remove the collapsed embolic protection filter 35 from the patient.

The delivery catheter 200-300 may be made of one or more polymers, such as, for example, a polyamide (e.g., nylon), fluorocarbon (e.g., polytetrafluoroethylene (PTFE)), polyether block amide (PEBA), polyolefin, or polyimide. As previously described, the catheter may further include a metallic (e.g., stainless steel) reinforcement structure 60 embedded within the one or more polymers to impart kink resistance and column strength to the catheter. Conventional catheter manufacturing methods known in the art, including, for example, extrusion and/or molding, may be employed to fabricate the catheter 200-300.

A delivery system 5 for an embolic protection device 10 has been described herein. The delivery system 5 may allow the embolic protection device 10 to be transported and deployed within vessels having a tortuous anatomy, making it possible to carry out carotid stenting procedures under a wider range of circumstances.

Although the present invention has been described with reference to certain embodiments thereof, other embodiments are possible without departing from the teachings herein.

What is claimed is:

1. An introducer for delivering an implant or other medical device within a patient, comprising:
a delivery catheter including a distal end and a proximal end; the catheter including a lumen for receiving an implant or medical device for delivery; and an actuator lumen; and
an actuator device located within the actuator lumen and operable to cause deflection of the catheter upon deployment of the actuator device; wherein
the actuator device is an elongate flexible actuator element fixed to or proximate a distal end of the delivery catheter and able to be pulled in a proximal direction so as to cause deflection of the delivery catheter.

2. An introducer according to claim 1, wherein the delivery lumen and the actuator lumen form a common lumen within the delivery catheter.

3. An introducer according to claim 1, wherein the delivery lumen and the actuator lumen are separate lumens within the delivery catheter.

4. An introducer according to claim 1, wherein the actuator advice is a wire.

5. An introducer according to claim 1, wherein the delivery catheter includes a guidewire lumen.

6. An introducer according to claim 1, wherein the delivery catheter includes a contrast agent lumen.

7. An introducer according to claim 1, including a supporting element at or proximate an outer surface of the delivery catheter.

8. An introducer according to claim 7, wherein the support element includes one of a braid, a mesh and a coil.

9. An introducer according to claim 1, wherein the delivery catheter includes a rapid exchange aperture therein.

10. An introducer for delivering an implant or other medical device within a patient, comprising:
a delivery catheter including a distal end and a proximal end, the catheter including a delivery lumen for receiving an implant or medical device for delivery; and an actuator lumen;
an elongate actuator wire located within the actuator lumen at a position offset from a centre axis of the delivery catheter and fixed at one of at or proximate the distal end of the delivery catheter and extending beyond the proximal end of the delivery catheter;
the elongate actuator element being able to be pulled in a proximal direction from the proximal end of the delivery catheter so as to cause deflection of the delivery catheter.
